(19) United States
(12) Patent Application Publication
(10) Pub. No.: US 2023/0070062 A1
(43) Pub. Date: Mar. 9, 2023

(54) METHOD AND SYSTEM, USING AN AI MODEL, FOR IDENTIFYING AND PREDICTING OPTIMAL FETAL IMAGES FOR GENERATING AN ULTRASOUND MULTIMEDIA PRODUCT

(71) Applicant: Clarius Mobile Health Corp., Vancouver (CA)

(72) Inventors: Laurent Pelissier, North Vancouver (CA); Kris Dickie, Vancouver (CA); Nishant Uniyal, Vancouver (CA); Jacob Ruben Barrera Loredo, Vancouver (CA); Parisa Asgharzadeh, Vancouver (CA)

(21) Appl. No.: 17/898,272

(22) Filed: Aug. 29, 2022

Related U.S. Application Data
(60) Provisional application No. 63/238,070, filed on Aug. 27, 2021.

(51) Int. Cl. G06V 10/774; A61B 8/08; A61B 8/00; G06T 7/62; G01S 15/89

(52) U.S. Cl. CPC G06V 10/774; A61B 8/0866; A61B 8/5223; A61B 8/565; A61B 8/463; G06T 7/62; G01S 15/8934; G06T 2207/10132; G06T 2207/20081; G06T 2207/30044

(57) ABSTRACT

A multi-media product is created from fetal ultrasound images, during scanning of a fetus using an ultrasound scanner, and employs a specifically trained artificial intelligence (AI) model to execute on a computing device communicably connected to an ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus and wherein after acquiring a new fetal ultrasound image during ultrasound scanning, the AI model selects the one or more fetal anatomical features, in whole or part, which are visually appealing for entertainment and keepsake purposes and are not part of a clinical assessment of the health or growth of the fetus and those selected non-clinical images are then used to generate the entertainment focused multi-media product.

### CROSS REFERENCE

This application claims priority from of U.S. Provisional Patent Application No. 63/238,070, filed Aug. 27, 2021.

### FIELD

The present disclosure relates generally to ultrasound imaging, and in particular, to systems and methods for generating an ultrasound multimedia product.

### BACKGROUND

Ultrasound is commonly used in medical examinations. For example, obstetrics examinations typically involve ultrasound scans of a fetus. These scans produce media items (e.g., images, videos, cineloops) interpreted by medical professionals to assess the development of the fetus. Since these scans usually provide the first images of an unborn baby, the media items may carry particular emotional meaning for parents.

Given the nature of ultrasound media items, it may be difficult for parents to interpret them. To make the ultrasound media items more digestible, some traditional systems allow users to manually select the media items from an obstetrics examination for the purpose of combining with audio, visual or text effects to generate a multimedia product. Using these traditional systems, the multimedia product may be written to physical media (such as a Compact Disc-Recordable (CD-R) or a Digital Video Disc (DVD)) or may be made available online.

Using these traditional methods to generate an ultrasound multimedia product is cumbersome. Manual selection of desirable media items, audio, and/or text overlays may be required prior to a multimedia product being generated. In some cases, manual effort is also required to transfer the media items to a computer where the multimedia product can be generated.

There is thus a need for improved systems and methods for generating an ultrasound multimedia product. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1 is a schematic diagram of the training and deployment of an AI model, according to an embodiment of the present invention.

FIG. 2 is flowchart diagram of the steps for training the AI model, according to an embodiment of the present invention.

FIG. 3A shows a user interface showing example fetal abdominal ultrasound images from an obstetrics examination, in accordance with at least one embodiment of the present invention.

FIG. 3B shows a user interface showing example fetal head ultrasound images from an obstetrics examination, in accordance with at least one embodiment of the present invention.

FIG. 3C shows a user interface showing example fetal femur abdominal ultrasound images from an obstetrics examination, in accordance with at least one embodiment of the present invention.

FIG. 4 is a flowchart diagram of an example method of image acquisition, prediction, identification and media product creation, according to an embodiment of the present invention.

FIG. 5 is an ultrasound image of an entire body of a fetus, from a side view.

FIG. 6A is an ultrasound image of two feet of a fetus.

FIG. 6B is an ultrasound image of one hand and partial arm of a fetus.

FIG. 7 is a schematic diagram of an ultrasound imaging system, according to an embodiment of the present invention.

FIG. 8 is a schematic diagram of a system with multiple ultrasound scanners, according to an embodiment of the present invention.

### DETAILED DESCRIPTION

The system of the present invention uses a transducer (a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

In a first broad aspect of the present disclosure, there are provided ultrasound systems and ultrasound-based methods for generating an ultrasound media product which comprise and enable the step of employing an AI model to identify one or more optimal fetal images for generating an ultrasound media product. In one embodiment, the AI model of the present invention is trained on a plurality of ultrasound images of fetal anatomy (whole and parts) to identify and select those specific fetal ultrasound images which are most visually appropriate and/or appealing for the creation of the media product. This media product, as defined further herein, is prepared for and directed to entertainment and keepsake purposes, as opposed to clinical and obstetric analyses.

At a high level, the present embodiments are generally directed to an automated way to select the most visually appropriate and/or appealing fetal ultrasound images for the creation of a media product, from a plurality of ultrasound images. The embodiments automate the act of such image identification and selection and remove the time-consuming steps typically performed manually by an operator, who, in environment of ultrasound procedures for entertainment/keepsake media production, may not be an experienced sonographer. As such, some embodiments herein provide for the creation of a “virtual sonographer”, for annotating, captioning and/or labelling ultrasound images for inclusion in a media product.

The embodiments herein generally provide ultrasound systems, ultrasound-based methods, computer-readable media storing computer-readable instructions, and portable computing devices for: i) identifying and selecting specific fetal ultrasound images which are most visually appropriate and/or appealing for the creation of the media product, by employing an AI model trained on a plurality of ultrasound images of fetal anatomy (whole and parts); ii) using one or more measurements from fetal ultrasound images, for example, head circumference (HC), crown rump length (CRL), humerus length (HL), radius length (RL), ulna length (UL), tibia length (TL), biparietal diameter (BPD), and abdominal circumference (AC), to auto-label fetal ultrasound image data and thereafter to train an AI model of the invention; iii) creating a “virtual sonographer”, for annotating, captioning and labelling fetal ultrasound images for inclusion in a media product; and iv) identifying and selecting the most visually appropriate and/or appealing fetal ultrasound images, using the AI model of the invention and subsequently passing such selected fetal ultrasound images (including for example, annotations, captions and labels) through a video rendering program to create a media product.

In present invention, the AI model is trained on a plurality of ultrasound images of fetal anatomy (whole fetus and/or parts thereof, for example specific anatomical features selected from the group consisting of: heart, arm, leg, face, head, brain, spine, kidney, liver, sexual organ, digits, belly, feet and hand). These training images enable the AI model to identify and select specific fetal ultrasound images which are most visually appropriate and/or appealing for the creation of a media product. Making a media product using a plurality of clinical images is less than desirable, as an experienced sonographer or radiologist (in a clinical setting) is looking for very specific medically-driven images, which are often not identifiable and hold no visual appeal to a parent. In contrast, the AI model of the invention is trained on robust variations of fetal ultrasound image data, including those images which would not “pass” as appropriate clinical images.

The present invention provides, in one aspect, a method of generating a multi-media product from fetal ultrasound images, during scanning of a fetus using an ultrasound scanner, comprising deploying an artificial intelligence (AI) model to execute on a computing device communicably connected to the ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquiring, at the computing device, a new fetal ultrasound image during ultrasound scanning; processing, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and incorporating ultrasound images comprising the selected fetal anatomical features into the multi-media product.

The present invention provides, in another aspect an ultrasound system for generating a multi-media product from fetal ultrasound images, comprising: an ultrasound scanner configured to acquire a plurality of new ultrasound frames; a processor that is communicatively connected to the ultrasound scanner and configured to process each new ultrasound frame of a plurality of new ultrasound frames against an artificial intelligence (“AI”) model, wherein said AI model is trained so that when the AI model is deployed, the computing device identifies and selects one or more fetal anatomical features, in whole or part, imaged in fetal ultrasound imaging data generated during ultrasound scanning, as part of a clinical exam of the fetus, wherein the selected one or more fetal anatomical features are visually appealing for entertainment and keepsake purposes and are not part of a clinical assessment of the health or growth of the fetus; acquire the new ultrasound image during ultrasound scanning; process, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and incorporate ultrasound images comprising the selected fetal anatomical features into the multi-media product.

The present invention provides, in another aspect a computer-readable media storing computer-readable instructions, for execution by at least one processor, wherein when the instructions are executed by the at least one processor, the at least one processor is configured to process a new ultrasound frame of a plurality of new ultrasound frames against an artificial intelligence (“AI”) model, wherein said AI model is trained so that when the AI model is deployed, a computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquire the new ultrasound image during ultrasound scanning; process, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and incorporate ultrasound images comprising the selected fetal anatomical features into the multi-media product.

The present invention provides, in another aspect an user interface communicatively associated with a processor, wherein the processor is configured to process a new ultrasound frame of a plurality of new ultrasound frames against an artificial intelligence (“AI”) model, wherein said AI model is trained so that when the AI model is deployed, a computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquire the new ultrasound image during ultrasound scanning; process, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and incorporate ultrasound images comprising the selected fetal anatomical features into the multi-media product and wherein said user interface enables a user to oversee, direct and control one or more actions and functions of the processor.

The present invention provides, in another aspect, an artificial intelligence (AI) model deployable on a computing device which is communicably connected to an ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of a fetus, wherein selected one or more fetal anatomical features are visually appealing for entertainment and keepsake purposes and are not part of a clinical assessment of the health or growth of the fetus; acquires, a new fetal ultrasound image during ultrasound scanning; processes, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and incorporates ultrasound images comprising the selected fetal anatomical features into a multi-media product.

The present invention provides, in another aspect, a multi-media product generated from fetal ultrasound images acquired during scanning of a fetus using an ultrasound scanner, wherein an artificial intelligence (AI) model is deployed to execute on a computing device communicably connected to the ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquiring, at the computing device, a new fetal ultrasound image during ultrasound scanning; processing, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and incorporating ultrasound images comprising the selected fetal anatomical features to form the multi-media product.

The present invention provides, in another aspect, a method of training an artificial intelligence (AI) model which is deployed to execute on a computing device communicably connected to the ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquiring, at the computing device, a new fetal ultrasound image during ultrasound scanning; processing, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and incorporating ultrasound images comprising the selected fetal anatomical features to form the multi-media product.

It is to be understood that “media product” (used interchangeably herein with “multimedia product”) as used herein comprises images, and/or videos and/or cineloops and which is rendered using a media rendering program/system, using ultrasound images generated (and optionally labelled, annotated and captioned) using the AI model of the present invention. The media product may be a video written to a physical media (such as a Compact Disc-Recordable (CD-r) or a Digital Video Disc (DVD) or made available online through cloud-based storage, through electronic communication or other transfer and data sharing means. Such media product may comprise audio, annotations, special effects (for example, frames and borders), image and/or text overlays. In some embodiments, the media product may comprise a plurality of cineloops.

Further, it is to be understood that the media/multi-media product may comprise a plurality of “AI model selected” 2D ultrasound images in accordance with the method and system of the invention. Alternatively, the media/multi-media product may comprise a 3D representation of one or more selected fetal anatomical features, formed of a plurality of 2D ultrasound images selected by the AI model of the invention. Such a 3D representation may be created (by way of example but not exclusively) using the methods described in U.S. patent application Ser. No. 16/995,712, filed Aug. 17, 2020, the entire contents of which are incorporated herein by reference.

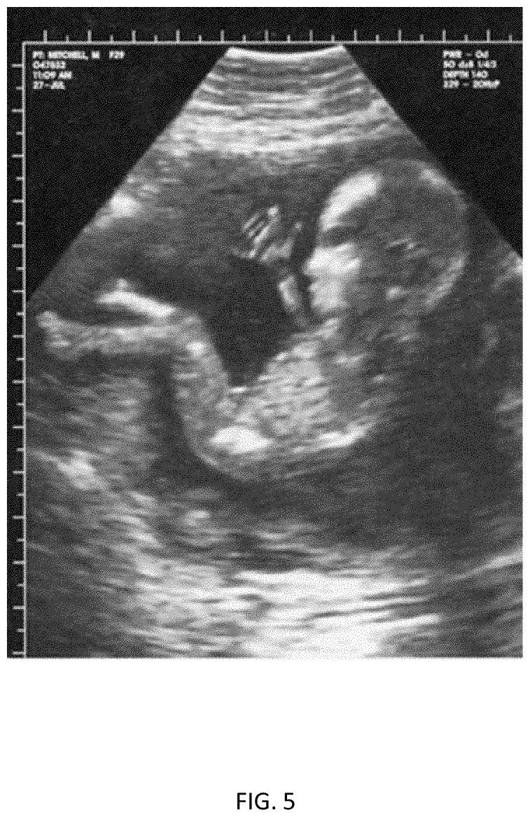

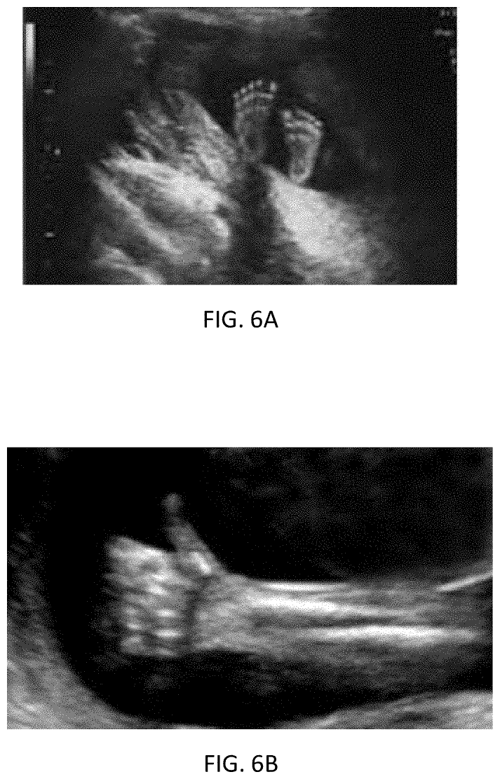

It is to be understood that ultrasound images are identified and selected by the trained AI model, in accordance with the method and system of the invention, around use-specific parameters, namely that one or more fetal anatomical features depicted in the images are visually appealing for entertainment and keepsake purposes and are not part of a clinical assessment of the health, growth or diagnostic issues of or relating to the fetus. In other words, these ultrasound images and one or more fetal anatomical features depicted in the images are selected by a uniquely and purpose trained AI model, to offer entertainment value, for inclusion in a non-clinical product. For example, when a non-medically trained person generally views an ultrasound screen during scanning of a fetus, most parts of the fetus are non-identifiable and are strange looking. As such, there is no human or emotional connection made to the images that are most critical from a clinician's perspective. So, while a plurality of images is captured during clinical ultrasound scanning of a fetus, many of such images are only clinically relevant. The AI model in accordance with the method and system of the invention, from this plurality of “clinically acquired” images, identifies, and selects substantially only those images (such as, for example, FIGS. 5, 6A and 6B) which are visually appealing for entertainment and keepsake purposes and are not part of a clinical assessment of the health, growth or diagnostic assessment of or relating to the fetus. Such images selected by the AI model may show all of parts of a fetus, from varying angles and orientations.

The embodiments herein further provide a method for the identification of specific fetal ultrasound images which are most visually appropriate and/or appealing for the creation of a media product, in ultrasound imaging data, by deploying an artificial intelligence (AI) model to execute on a computing device, wherein the AI model is trained to identify a plurality of whole fetus and/or fetal parts thereof, imaged in ultrasound imaging data, and when deployed, the computing device generates a selection, from new fetal ultrasound images, of those which are most visually appropriate and/or appealing for the creation of a media.

In some embodiments, the ultrasound frames of a new ultrasound images, imaged in ultrasound imaging data may be processed against an artificial intelligence (AI) model on a per pixel basis, and the selection/prediction that a type of fetal image of the plurality of different types of fetal images is imaged in new ultrasound imaging data may be generated on a per pixel basis. When deployed, an output of the AI model for a first pixel of the new ultrasound imaging data may be used to corroborate the output of the AI model for a second pixel of the new ultrasound imaging data adjacent or within the proximity to the first pixel.

Alternatively, the ultrasound frames of new ultrasound images, imaged in ultrasound imaging data may be processed against an artificial intelligence (AI) model on a line/sample basis, and the selection/prediction that a type of fetal image of the plurality of different types of fetal images is imaged in new ultrasound imaging data may be generated on a line/sample basis.

Within the scope of the present invention, an AI model is trained to identify a plurality of different components of fetal anatomy (from a whole fetus, in various views and orientations and/or one or more parts thereof) imaged in ultrasound imaging data, and when deployed, a processor with at least one computing device identifies and selects from within the plurality of images in new ultrasound imaging data, of different components of fetal anatomy, in whole or in part, that are most visually appropriate and/or appealing for the creation of a media product, such being referred to as the “selected fetal ultrasound images”.

In another broad aspect of the present disclosure, there is provided a server including at least one processor and at least one memory storing instructions for execution by the at least one processor, wherein when executed, the instructions cause the at least one processor to: process each new ultrasound frame of the plurality of new ultrasound frames against an artificial intelligence (“AI”) model, wherein said AI model is trained to identify different components of fetal anatomy, in whole or in part, that are most visually appropriate and/or appealing for the creation of a media product (the ‘selected fetal ultrasound images”), as imaged in existing ultrasound imaging data; acquire the plurality of new ultrasound frames; using the AI model identify one or more fetal ultrasound images are imaged in new ultrasound frames and passing such identified fetal ultrasound images (including for example, annotations, captions and labels) through a video rendering program to create a media product.

In another broad aspect of the present disclosure, there is provided a computing device comprising at least one processor and at least one memory storing instructions for execution by the at least one processor, wherein when executed, the instructions cause the at least one processor to: process each new ultrasound frame of the plurality of new ultrasound frames against an artificial intelligence (“AI”) model, wherein said AI model is trained to identify different components of fetal anatomy, in whole or in part, that are most visually appropriate and/or appealing for the creation of a media product (the “selected fetal ultrasound images”), as imaged in existing ultrasound imaging data; acquire the plurality of new ultrasound frames; using the AI model identify one or more fetal ultrasound images are imaged in new ultrasound frames and passing such identified fetal ultrasound images (including for example, annotations, captions and labels) through a video rendering program to create a media product.

In another broad aspect of the present disclosure, there is provided a computer readable medium storing instructions for execution by at least one processor, wherein when the instructions are executed by the at least one processor, the at least one processor is configured to: process each new ultrasound frame of the plurality of new ultrasound frames against an artificial intelligence (“AI”) model, wherein said AI model is trained to identify different components of fetal anatomy, in whole or in part, that are most visually appropriate and/or appealing for the creation of a media product (the ‘selected fetal ultrasound images”), as imaged in existing ultrasound imaging data; acquire the plurality of new ultrasound frames; using the AI model identify one or more fetal ultrasound images are imaged in new ultrasound frames and passing such identified fetal ultrasound images (including for example, annotations, captions and labels) through a video rendering program to create a media product.

Within the scope of the invention, the selected fetal ultrasound images, which the AI model is trained to identify may, in some instances, not be clinically ideal images or ones which would be obstetrically useful. The goal of image identification and labeling, for the purpose of the collective fetal image AI model of the invention, is entertainment and keepsake media production, and images which are usable for this purpose. As such, the AI model may be uniquely trained for this purpose as described herein.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

Building a fetal image specific AI model and using such a model to optimize images used in the creation of a media product for entertainment/keepsake purposes has not previously been undertaken or the benefits thereof appreciated. Further details, embodiments, and features are described herein.

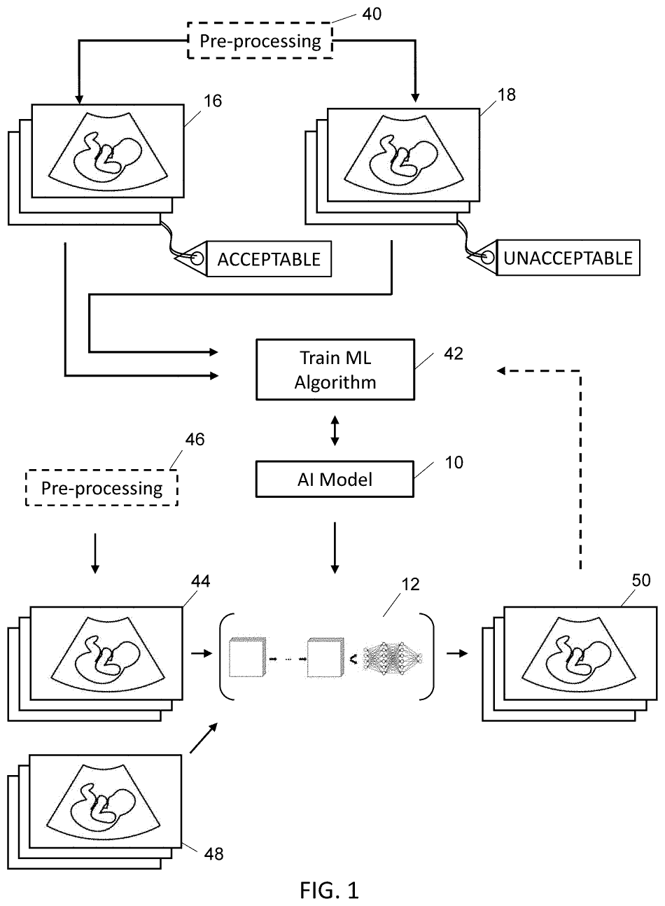

Referring to FIG. 1, shown there generally is a schematic diagram of a training and deployment of an AI model 10. According to an embodiment of the present invention, there is shown a method of training a neural network 12 to identify a plurality of fetal anatomical parts (from entire fetus to parts thereof), wherein each fetal part is depicted by a plurality of ultrasound images. Specifically, during use and deployment, neural network 12 identifies one or more optimal fetal images for generating an ultrasound media product.

For training, a number of ultrasound frames of a fetus (in whole view, from varying perspectives and parts thereof) may be acquired using an ultrasound scanner (hereinafter “scanner”, “probe”, or “transducer” for brevity). The ultrasound frames may be acquired by fanning a series of a planes (with a frame each containing a sequence of transmitted and received ultrasound signals), through an angle and capturing a different ultrasound frame at each of a number of different angles. During the scanning, the scanner may be held steady by an operator of the scanner while a motor in the head of the scanner tilts the ultrasonic transducer to acquire ultrasound frames at different angles. Additionally or alternatively, other methods of acquiring a series of ultrasound frames may be employed, such as using a motor to translate (e.g., slide) the ultrasonic transducer or rotate it, or manually tilting, translating or rotating the ultrasound scanner. When acquiring the number of ultrasound frames, it is possible, but not necessary, to initially place the scanner in the midsagittal plane of the fetus.

The AI model if preferably trained with a robust selection of fetal images of varying views. For example, these different views may include coronal and/or transverse plane views of the fetus, including views from different angles that combine any of a sagittal plane view, a coronal plane view, or a transverse plane view. In these embodiments, the scanner may be placed in an arbitrary orientation with respect to the fetus, provided that the scanner captures at least a portion of the fetus.

In some embodiments, the fetal ultrasound scan to acquire images may be performed for a regular obstetrics examination during a pregnancy. As will be understood by persons skilled in the art, there are several standard views of a fetus that may be considered part of a standard ultrasound scan during pregnancy. These views may allow medical professionals to determine whether the growth of the unborn baby is proceeding as expected. During the scans, images may be obtained; however, for training of the AI model of the invention, non-clinically useful or acceptable images may also be used.

Referring still to FIG. 1, training ultrasound frames (16 and 18) may include ultrasound frames with features that are tagged as acceptable (16) and representative of specific fetal characteristics which are most visually appropriate and/or appealing for the creation of the media product and/or ultrasound frames that are tagged respectively as unacceptable (18) and unrepresentative of specific fetal characteristics which are most visually appropriate and/or appealing for the creation of the media product.

Both the training ultrasound frames labeled as Acceptable and Unacceptable, for each particular fetus (whole or part), may themselves be used for training and/or reinforcing AI model 10. This is shown in FIG. 1 with tracking lines from both 16 and 18 to training algorithm step 42.

In some embodiments, an optional pre-processing act 40 may be performed on the underlying ultrasound image frames 16 and 18 to facilitate improved performance and/or accuracy when training the machine learning (ML) algorithm. For example, it may be possible to pre-process the ultrasound images 16 and 18 through a high contrast filter to reduce the granularity of greyscale on the ultrasound images 16 and 18.

Additionally, or alternatively, it may be possible to reduce scale of the ultrasound images 16 and 18 prior to providing the ultrasound images 16 and 18 to the training algorithm step 42. Reducing the scale of ultrasound images 16 and 18 as a preprocessing step may reduce the amount of image data to be processed during the training act 42, and thus may reduce the corresponding computing resources required for the training act 42 and/or improve the speed of the training act 42.

Various additional or alternative pre-processing acts may be performed in act 40. For example, these acts may include data normalization to ensure that the various ultrasound frames 16 and 18 used for training have generally the same dimensions and parameters.

Referring still to FIG. 1, the various training frames 16 and 18 may, at act 42, be used to train a ML algorithm. For example, the various training ultrasound frames 16 and 18, may be inputted into deep neural network 12 that can learn how to predict desired fetal characteristics for media product creation of new ultrasound images as compared to all trained and stored fetal images. For example, the neural network may learn to detect the most desired fetal characteristics and to discard the presence of differing nearby anatomical features which would decrease the quality of the media product.

The result of the training may be the AI model 10, which represents the mathematical values, weights and/or parameters learned by the deep neural network to predict desired fetal characteristics for media product creation of new ultrasound images as compared to all trained and stored fetal images. The training act 42 may involve various additional acts (not shown) to generate a suitable AI model 10. For example, these various deep learning techniques such as regression, classification, feature extraction, and the like. Any generated AI models may be iteratively tested to ensure they are not overfitted and sufficiently generalized for creating the comparison and list of probabilities in accordance with method of the invention.

In some embodiments, using a cross-validation method on the training process would optimize neural network hyper-parameters to try to ensure that the neural network can sufficiently learn the distribution of all possible fetal image types without overfitting to the training data. In some embodiments, after finalizing the neural network architecture, the neural network may be trained on all of the data available in the training image files.

In various embodiments, batch training may be used and each batch may consist of multiple images, thirty-two for example, wherein each example image may be gray-scale, preferably 128*128 pixels although 256*256 pixels and other scaled may be used, without any preprocessing applied to it.

In some embodiments, the deep neural network parameters may be optimized using the Adam optimizer with hyper-parameters as suggested by Kingma, D. P., Ba, J. L.: Adam: a Method for Stochastic Optimization, International Conference on Learning Representations 2015 pp. 1-15 (2015), the entire contents of which are incorporated herewith. The weight of the convolutional layers may be initialized randomly from a zero-mean Gaussian distribution. In some embodiments, the Keras™ deep learning library with TensorFlow™ backend may be used to train and test the models.

In some embodiments, during training, many steps may be taken to stabilize learning and prevent the model from over-fitting. Using the regularization method, e.g., adding a penalty term to the loss function, has made it possible to prevent the coefficients or weights from getting too large. Another method to tackle the over-fitting problem is dropout. Dropout layers limit the co-adaptation of the feature extracting blocks by removing some random units from the neurons in the previous layer of the neural network based on the probability parameter of the dropout layer. Moreover, this approach forces the neurons to follow overall behaviour. This implies that removing the units would result in a change in the neural network architecture in each training step. In other words, a dropout layer performs similar to adding random noise to hidden layers of the model. A dropout layer with the dropout probability of 0.5 may be used after the pooling layers.

Data augmentation is another approach to prevent over-fitting and add more transitional invariance to the model. Therefore, in some embodiments, the training images may be augmented on-the-fly while training. In every mini-batch, each sample may be translated horizontally and vertically, rotated and/or zoomed, for example. The present invention is not intended to be limited to any one particular form of data augmentation, in training the AI model. As such, any mode of data augmentation which enhances the size and quality of the data set, and applies random transformations which do not change the appropriateness of the label assignments may be employed, including but not limited to image flipping, rotation, translations, zooming, skewing, and elastic deformations.

Referring still to FIG. 1, after training has been completed, the sets of parameters stored in the storage memory may represent a trained neural network of a plurality of fetal images (in whole and parts thereof) to then predict and identify desired fetal characteristics for media product creation of new ultrasound images as compared to all trained and stored fetal images.

In order to assess the performance of AI model 10, the stored model parameter values can be retrieved any time to perform image assessment through applying an image to the neural networks (shown as 12) represented thereby. In some embodiments, the deep neural network may include various layers such as convolutional layers, pooling layers, and fully connected layers. In some embodiments, the final layers may include a softmax layer as an output layer having outputs which eventually would demonstrate respective determinations that an input set of pixels fall within a particular area above or below a fetus (or part thereof) boundary, in the training images. Accordingly, in some embodiments, the neural network may take at least one image as an input and output a binary mask indicating which pixels belong to the area above a fetus (or part thereof) boundary (e.g., the AI model classifies which area each pixel belongs to).

To increase the robustness of the AI model 10, in some embodiments, a broad set of training data may be used at act 42. For example, it is desired that ultrasound images of a plurality of different fetuses, in whole and a variety of parts thereof, from views including but not limited to coronal and/or transverse plane views, including views from different angles that combine any of a sagittal plane view, a coronal plane view, or a transverse plane view.

More specifically, training images 16-18 may be labeled with one or more features associated with/are hallmarks of a selected part of a fetus. This may include identifying a variety of features visualized in the captured training image. In at least some embodiments, this data may be received from trainer/user input. For example, a trainer/user may label the features relevant for the application visualized in each training image.

The image labeling can be performed, for example, by a trainer/user observing the training ultrasound images, via a display screen of a computing device, and manually annotating the image via a user interface. In some aspects, the training ultrasound images used for the method herein will only be images in which the image quality is of a sufficient quality threshold to allow for proper and accurate feature identification. For example, this can include training ultrasound images having a quality ranging from a minimum quality in which target features are just barely visible for labelling (e.g., annotating), to excellent quality images in which the target features are easily identifiable. In various embodiments, the training medical images can have different degrees of images brightness, speckle measurement and SNR. Accordingly, training ultrasound images 16 and 18 can include a graduation of training images ranging from images with just sufficient image quality to high image quality. In this manner, the machine learning model may be trained to identify features on training medical images that have varying levels of sufficient image quality for later interpretation and probability assessment. As noted above, an advantage of the AI model of the invention is the value of optional training on non-standard/non-medically compliant images which may be more suited to media product creation, from a visual or aesthetic perspective. By way of example only, FIGS. 5, 6A and 6B (full body, feet, hands) are illustrative of easily identifiable fetal ultrasound images which may be desirable for an entertainment/keepsake media product.

Overall, the scope of the invention and accorded claims are not intended to be limited to any one particular process of training AI model 10. Such examples are provided herein by way of example only. AI model 10 may be trained by both supervised and unsupervised learning approaches although due to scalability, unsupervised learning approaches, which are well known in the art, are preferred. Other approaches may be employed to strengthen AI model 10.

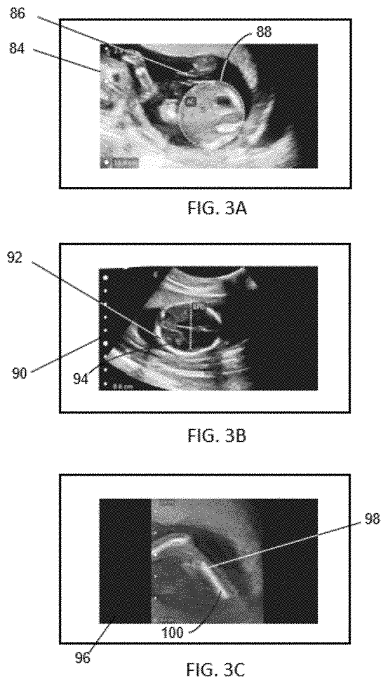

For example, and with reference to FIGS. 3A, 3B and 3C, AI Model 10 may be trained with automatically labelled fetal biometric measurements, including but not limited to head circumference (HC), crown rump length (CRL), humerus length (HL), radius length (RL), ulna length (UL), femur length (FL), tibia length (TL), biparietal diameter (BPD), and abdominal circumference (AC), such biometric data assisting in accurate fetal anatomy AI model training. FIG. 3A shows ultrasound image 84 of abdomen 86 upon which is overlayed dotted markers of abdominal circumference (AC) 88. FIG. 3B shows ultrasound image 90 of fetal head 92 upon which is overlayed dotted markers of a biparietal diameter (BPD) 94. FIG. 3C shows ultrasound image 96 of fetal femur 98 upon which is overlayed dotted markers of a femur length 100.

In one aspect, on an ultrasound user interface, functions relating to fetal feature measurement may be carried out by simple touch interactions. For instance, the position of the measurement calipers can be easily adjusted by a drag gesture. Other measurement methods range from semi- to fully automatic, with little to no user input. All such methods are fully known within the art and may be employed within the present invention to acquire and label fetal biometric measurements, for AI model training.

Turning back to FIG. 1, once a satisfactory AI model 10 is generated, the AI model 10 may be deployed for execution on a neural network 12 to identify and select optimal and desired fetal characteristics for media product creation on new ultrasound images 44 stored fetal images 48. Notably, the neural network 12 is shown in FIG. 1 for illustration as a convolution neural network—with various nodes in the input layer, hidden layers, and output layers. However, in various embodiments, different arrangements of the neural network 12 may be possible.

In various embodiments, prior to being processed for analysis as described herein, the new ultrasound images 44 may optionally be pre-processed. This is shown in FIG. 1 with the pre-processing act 46 in dotted outline. In some embodiments, these pre-processing acts 46 may be analogous to the pre-processing acts 40 performed on the training ultrasound frames 16 and 18 (e.g., processing through a high contrast filter and/or scaling), to facilitate improve accuracy in identifying and selecting optimal and desired fetal characteristics for media product creation.

In various embodiments, the new fetal ultrasound images 44 may be live images acquired by an ultrasound imaging system (e.g., the system discussed with respect to FIGS. 8 and 9). For example, the AI model 10 may be deployed for execution on the scanner 131 and/or the display device 150 discussed in more detail below. Additionally or alternatively, the AI model 10 may be executed on stored images 48 that were previously acquired (e.g., as may be stored on a Picturing Archiving and Communication System (PACS)). When executed in this manner, the AI model 10 may allow the neural network 12 to predict optimal and desired fetal characteristics for media product creation on the new ultrasound frames 44, resulting in a series of ultrasound frames 50 with predicted optimal and desired fetal characteristics for media product creation.

In some embodiments, the ultrasound frames 50 with predicted optimal and desired fetal characteristics for media product creation may optionally each be labeled as either acceptable or unacceptable, and these labeled ultrasound frames may themselves be used for training and/or reinforcing the AI model 10 via training act 42. This is shown in FIG. 1 with a dotted line from the ultrasound frames 50 with the predicted cut lines being provided to the training act 42.

The training images file may include an image identifier field for storing a unique identifier for identifying an image included in the file, a segmentation mask field for storing an identifier for specifying the to-be-trimmed area, and an image data field for storing information representing the image.

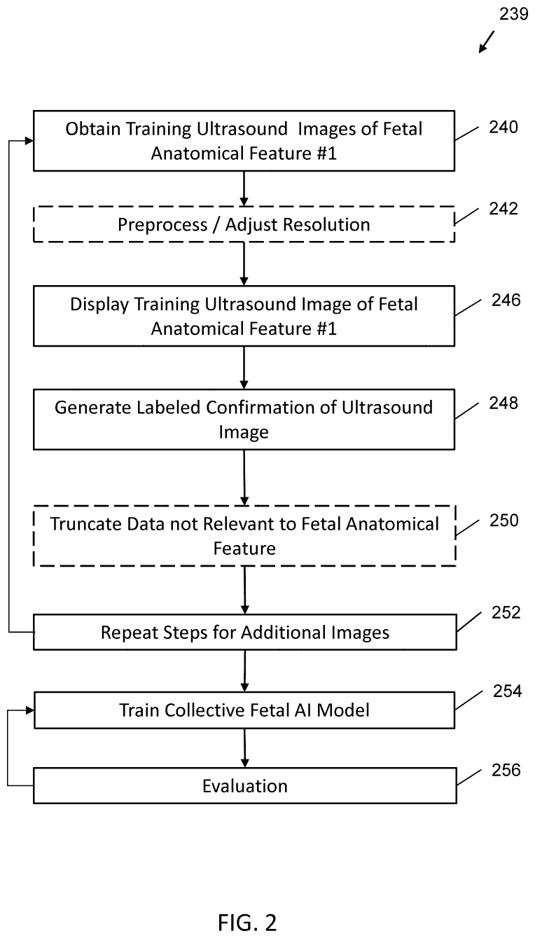

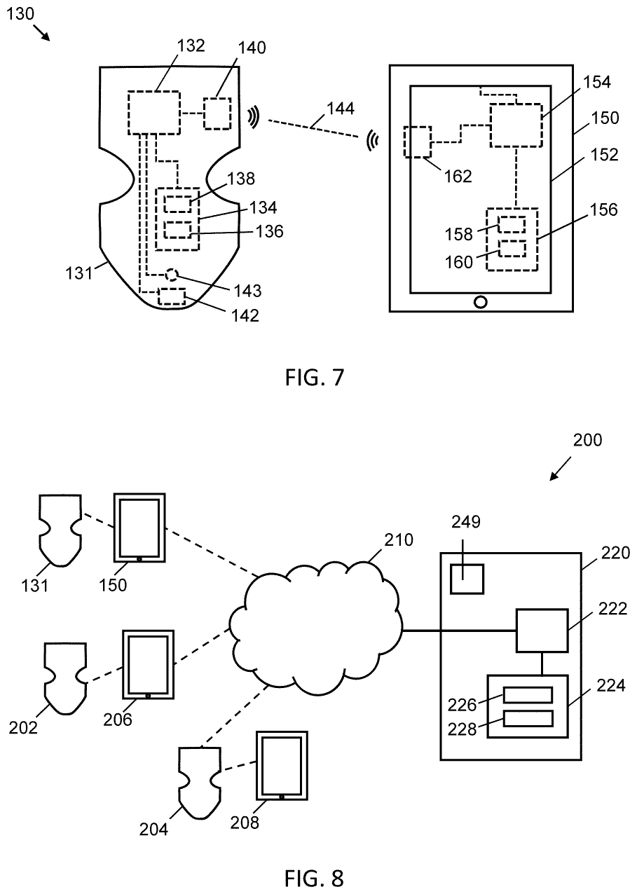

FIG. 2 is flowchart diagram of the steps for training the AI model of FIG. 1, according to an embodiment of the present invention. Method 239 is described below with regard to the systems and components depicted in FIGS. 7 and 8, though it should be appreciated that method 239 may be implemented with other systems and components without departing from the scope of the present disclosure. In some embodiments, method 239 may be implemented as executable instructions in any appropriate combination of the imaging system 130, for example, an external computing device connected to the imaging system 130, in communication with the imaging system 130, and so on. As one example, method 239 may be implemented in non-transitory memory of a computing device, such as the controller (e.g., processor) of the imaging system 130 in FIG. 7. At 240, method 239 may include acquiring a dataset of sample images for training the neural network. Each sample image in the dataset may be a sample ultrasound image depicting a sample fetus (whole fetus or anatomical components thereof).

Referring still to FIG. 2, in step 240, a training ultrasound image may be obtained. For example, a training ultrasound image may be acquired by the scanner 131 (as shown in FIG. 7) transmitting and receiving ultrasound energy. The training ultrasound image may generally be a post-scan converted ultrasound image. While the method of FIG. 2 is described in relation to a single training ultrasound image, the method may also apply to the use of multiple training ultrasound images. While the method of FIG. 2 is described in relation to a post-scan ultrasound image, it is to be understood that pre-scan images, may be used, as described in U.S. patent application Ser. No. 17/187,851 filed Feb. 28, 2021, the entire contents of which are incorporated herein by reference.

Optionally, in step 242 (as shown in dotted outline), the resolution of the training ultrasound image may be adjusted. For example, the resolution may be increased or decreased. The purpose of this may be to provide the labeler (e.g., a medical professional with relevant clinical expertise) with training ultrasound images that have a more standardized appearance. This may help to maintain a higher consistency with which the labeler identifies anatomical features in the training ultrasound images. Besides the resolution, other parameters of the training ultrasound image may also be adjusted such as input scaling, screen size, pixel size, aspect ratio, and the removal of dead space, as described above (including, for example, data augmentation and other preprocessing steps).

In step 246, the training ultrasound image may be displayed on a display device, such as the display device 150 discussed in more detail below in relation to FIG. 7. The labeler can then identify a particular fetal anatomy in the training ultrasound image by, for example, tagging it with a name from a pull-down menu or by using other labeling techniques and modalities. The labeler then can mark the training ultrasound image around the particular fetal anatomy that the labeler has identified in the training ultrasound image. In step 246, the system that is used for the training may receive the identification of the fetal anatomy. In step 248, the system may generate, from the labeler's marking inputs, a labeled training ultrasound image, and display it on the display device. In various embodiments, steps 246 and 248 may readily be interchanged with each other. As described in FIGS. 3A, B and C, the generation of labeled confirmation at steps 248 may automatically proceed, without trainer intervention, using fetal measurement data which directs to the identity of a particular fetal anatomical feature.

Once the training ultrasound image has been marked and labeled, the system may then remove, in step 250, optionally, (as shown in dotted outline), regions of the labeled ultrasound data frame that are both outside the area of the identified fetal anatomy and outside areas relevant for the AI model to recognize the particular fetal anatomy. For example, the labeled ultrasound data frame may be truncated at one or more sides. Truncation of some of the ultrasound data may allow the training of the AI model to proceed more quickly. At step 252, there is provided a redirection to complete steps 240-250 a plurality of times, both for a first fetal anatomical feature (whole fetus or parts thereof) and subsequent fetal anatomical features (whole fetus or parts thereof) thereby to build a robust fetal-specific AI model, populated with images which are representative of specific fetal characteristics which are most visually appropriate and/or appealing for the creation of a media product. At step 254, the labeled raw ultrasound data frame is then used for training the AI model 10. At step 256, once training is completed, the AI model may be used to perform identifications and selections on an unseen dataset to validate its performance, such evaluation at step 256 feeding data back to train the AI model at step 254.

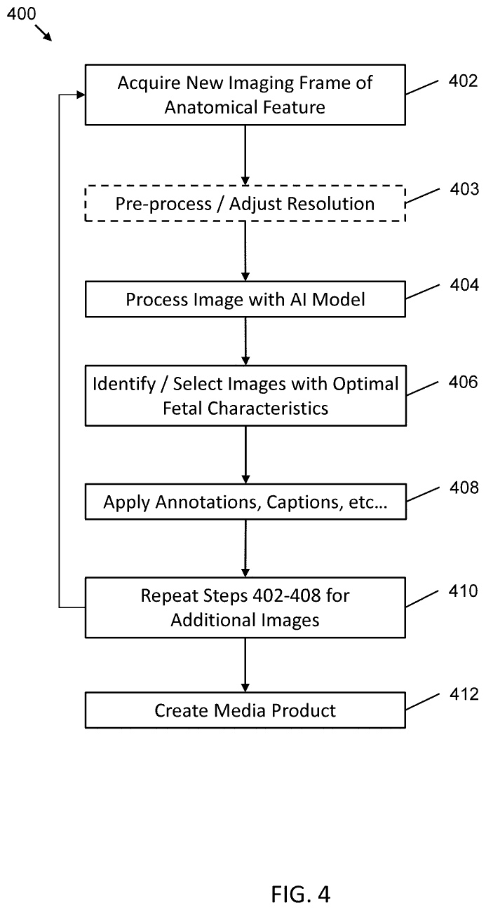

Referring to FIG. 4, a flowchart diagram of a method, generally indicated at 400, of new image acquisition of an anatomical feature, processing against an AI model and identification/selection of optimal fetal characteristics for media product creation, according to at least one embodiment of the present invention is shown. Method 400 further supports and aligns with the steps described above in regard to FIG. 2. At 402, the ultrasound imaging system (referred to in FIG. 7), may acquire ultrasound imaging data. For example, a user may operate an ultrasound scanner (hereinafter “scanner”, “probe”, or “transducer” for brevity) to capture images of a patient. The ultrasound frames (B-mode) may be acquired by acquiring a series of a images (with a frame each containing a sequence of transmitted and received ultrasound signals) of different views of a fetus.

Further, at step 403, new ultrasound imaging data may optionally be pre-processed and/or augmented as described above. The AI model 10: i) at step 404, processes new ultrasound imaging data; ii) at step 406, identifies optimal fetal characteristics for media product creation; and ii) at step 408, apply labels, annotations, text, captions such that the AI model collectively prepares a plurality of suitable image inputs for a media creation platform. At step 410, direction is provided to repeat steps 402-408 for additional new images and at step 412, using a media creation platform, a media product is prepared. As used herein, the term “virtual sonographer” refers to these AI-directed steps for annotating, captioning and labelling the (AI model) selected fetal ultrasound images for inclusion in a media product.

In various embodiments, the methods described herein for generating a multimedia product may be implemented using a graphics library such as the three.js library (which provides a JavaScript WebGL (Web Graphics Library) Application Programming Interface (API)). For example, a theme with a number of effects may be specified via a script that calls the available API calls in WebGL to set the order in which selected ultrasound media items are to be played, and/or to set various visual parameters of any chosen effects. In some embodiments, one or more WebGL scenes may be set up for a theme, and each scene may have an associated WebGL camera (which may be the same across one or more scenes). When applying the theme, various ultrasound media items marked for inclusion into the multimedia product may be included as objects and added to a given scene. In various embodiments, if there are more ultrasound media items marked for inclusion that available scenes in a theme, scenes may be reused when generating a multimedia product based on that given theme.

In one example embodiment, and with reference back to FIG. 8, the scanner 131 may be configured to display a screen 152 which provides a user-selectable option that, when selected, causes the scanner 131 to communicate with the server 220 to cause the methods for generating a multimedia product discussed herein to be performed. The server 220 may be configured to provide a multimedia product generator 249 to perform various acts of the methods discussed herein. The server 220 may be configured to communicate with the scanner 131 to receive and store ultrasound media items into a corresponding suitable storage mechanism such as database. The server 220 may also provide a multimedia product generator 249 that is configured to generate an ultrasound media product as discussed herein. In various embodiments, the multimedia product generator 249 may be provided in the form of software instructions (e.g., a script) configured to execute on server 220 and/or transmitted from server 220 to a viewing computing device 150 for execution thereon. As noted above, in one example embodiment, the software instructions may be provided in the form of a script written in a scripting language that can access the WebGL API.

Although illustrated as a single server in the block diagram of FIG. 8, the term “server” herein may encompass one or more servers such as may be provided by a suitable hosted storage and/or cloud computing service. Further, in various embodiments, the databases illustrated may not reside with the server 220. For example, the data may be stored on managed storage services accessible by the server 220 and/or the viewing computing device 150 executing a script.

In some embodiments, the server 220 may also be communicably coupled to a billing or accounting system for a professional associated with the scanner 131. In such embodiments, upon generating the multimedia product, the server 220 may communicate with the billing or accounting system so as to add a charge to a purchaser/patient for creation of the ultrasound multimedia product. In some embodiments, the server 220 may also be communicably coupled to an electronic delivery system such that the multimedia product may be provided directly (in some cases automatically upon creation, without user intervention) to the purchaser/patient. In other embodiments, a professional associated with the scanner 131, is provided with a workflow on-screen option to electronically deliver the multimedia product to the purchaser/patient.

The viewing computing device 150 can be any suitable computing device used to generate the multimedia product and/or access the multimedia product generated by the server 220. For example, in the embodiment where the multimedia product generator 249 of server 220 is provided in the form of a script that can make WebGL API calls, the script may be transmitted to the viewing computing device 150 so that the multimedia product may be generated when the script is executed in a browser. A graphics library (GL) engine may interpret the script and live render the multimedia product for viewing at the viewing computing device 150. In some embodiments, the live render of the multimedia product may involve processing by a Graphics Processing Unit (GPU) provided on the viewing computing device 150.

In some embodiments, the server 220 may be configured to execute WebGL API calls such that a script (or portion thereof) may be executed at the server 220 to perform pre-rendering. For example, this may allow for more flexible distribution of a generated multimedia product. For example, the multimedia product generator 249 may be configured to generate a standalone multimedia file (e.g., a Motion Picture Experts Group (MPEG)-4, or MP4 file) that can be transmitted from server 220 to the viewing computing device 150 for displaying thereon (e.g., for playing using a media player (not shown)). As used herein, the term “multimedia product” may refer to a pre-rendered multimedia experience (e.g., a generated video file) and/or any multimedia experience that is dynamically generated each time.

In various embodiments, the multimedia product may be configured to be interactive. For example, when a given ultrasound media item is displayed during the playback of a generated multimedia product, the multimedia product may be configured to receive user input to zoom in or otherwise highlight the ultrasound media item being displayed. Additionally or alternatively, the multimedia product may be configured to provide gallery controls on the display of various frames of the generated multimedia product. For example, these gallery controls may be configured to receive “next” or “previous” input during the display of a given ultrasound media item, so as to allow a user to advance forward to the next ultrasound media item, or navigate back to a previously-viewed ultrasound media item. In various embodiments, the interactivity may be implemented using API calls available in WebGL.

In some although not all embodiments of the invention, the multimedia product may be generated from ultrasound media images obtained during a regular medically-necessary examination. For example, in the case of fetal ultrasound scans, the ultrasound media images may be obtained during regular obstetrics examinations where medical professionals assess the health of the unborn baby. By using ultrasound media items from a medically-necessary examination to generate the multimedia product, the ALARA (As Low As Reasonably Achievable) principle can be followed with respect to avoiding unnecessary exposure to ultrasound energy.

While a number of exemplary aspects and embodiments have been discussed above, those of skill in the art will recognize that may be certain modifications, permutations, additions and sub-combinations thereof. While the above description contains many details of example embodiments, these should not be construed as essential limitations on the scope of any embodiment. Many other ramifications and variations are possible within the teachings of the various embodiments.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the use of the masculine can refer to masculine, feminine or both;
where numerical values are given, they are specified to the nearest significant figure;
the singular forms “a”, “an”, and “the” also include the meaning of any appropriate plural forms.
Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

The term “2D-mode” refers to any ultrasound imaging mode that provides a two-dimensional cross-sectional view of body tissue.

The term “B-mode” refers to the brightness mode of an ultrasound scanner, which displays the acoustic impedance of a two-dimensional cross-section of body tissue.

The term “AI model” means a mathematical or statistical model that may be generated through artificial intelligence techniques such as machine learning and/or deep learning. For example, these techniques may involve inputting labeled or classified data into a neural network algorithm for training, so as to generate a model that can make predictions or decisions on new data without being explicitly programmed to do so. Different software tools (e.g., TensorFlow™, PyTorch™, Keras™) may be used to perform machine learning processes.

The term “module” can refer to any component in this invention and to any or all of the features of the invention without limitation. A module may be a software, firmware or hardware module, and may be located, for example, in the ultrasound scanner, a display device or a server.

The term “communications network” can include both a mobile network and data network without limiting the term's meaning, and includes the use of wireless (e.g. 2G, 3G, 4G, 5G, WiFi™, WiMAX™, Wireless USB (Universal Serial Bus), Zigbee™, Bluetooth™ and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “operator” (or “user”) may refer to the person that is operating an ultrasound scanner (e.g., a clinician, medical personnel, a sonographer, ultrasound student, ultrasonographer and/or ultrasound technician).

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “scan convert”, “scan conversion”, or any of its grammatical forms refers to the construction of an ultrasound media, such as a still image or a video, from lines of ultrasound scan data representing echoes of ultrasound signals. Scan conversion may involve converting beams and/or vectors of acoustic scan data which are in polar (R-theta) coordinates to cartesian (X-Y) coordinates.

The term “system” when used herein, and not otherwise qualified, refers to an ultrasound imaging system, the system being a subject of the present invention. In various embodiments, the system may include an ultrasound machine (including a display and one or more transducers); an ultrasound scanner and a display device; and/or an ultrasound scanner, display device and a server. This includes systems for generating 2D and 3D representations.

The term “ultrasound image frame” (or “image frame” or “ultrasound frame”) refers to a frame of post-scan conversion data that is suitable for rendering an ultrasound image on a screen or other display device.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112(f) unless the words “means for” or “step for” are explicitly used in the particular claim.
It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

### Claim Support:

In a first broad aspect of the present disclosure, there is provided a method of generating a multi-media product from fetal ultrasound images, during scanning of a fetus using an ultrasound scanner, comprising: deploying an artificial intelligence (AI) model to execute on a computing device communicably connected to the ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; acquiring, at the computing device, a new fetal ultrasound image during ultrasound scanning; processing, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and incorporating ultrasound images comprising the selected fetal anatomical features into the multi-media product.

In some embodiments, the AI model is trained with a plurality of training ultrasound images comprising labelled fetal biometric measurements, generated by one of a manual, semi automatic means or fully automatic means.

In some embodiments, the AI model is trained with a plurality of training ultrasound images, comprising fetal anatomical features labeled and tagged from an identifier menu by one of a manual, semi automatic means or fully automatic means.

In some embodiments, the new fetal ultrasound image is one or a combination of a live acquired fetal ultrasound image and a stored, previously acquired fetal ultrasound image.

In some embodiments, fetal biometric measurements are selected from the group consisting of head circumference (HC), crown rump length (CRL), humerus length (HL), radius length (RL), femur length (FL), ulna length (UL), tibia length (TL), biparietal diameter (BPD), and abdominal circumference (AC), and wherein the method of training the AI model comprises identifying and labelling one or more fetal anatomical features in a fetal ultrasound image using the fetal biometric measurements.

In some embodiments, when identifying and selecting one or more fetal anatomical features, in whole or part, imaged in fetal ultrasound imaging data generated during ultrasound scanning, the AI model processes the fetal ultrasound imaging data on a per pixel basis, and the probability that a fetal anatomical feature is imaged in new ultrasound imaging is generated on a per pixel basis.

In some embodiments, when identifying and selecting one or more fetal anatomical features, in whole or part, imaged in fetal ultrasound imaging data generated during ultrasound scanning, the AI model processes the ultrasound imaging data on a per pixel basis, and the probability that a fetal anatomical feature is imaged in new ultrasound imaging is generated on a per pixel basis, and wherein, when deployed, an output of the AI model for a first pixel of the new ultrasound imaging data is used to corroborate the output of the AI model for a second pixel of the new ultrasound imaging data adjacent to the first pixel.

In some embodiments, when identifying and selecting one or more fetal anatomical features, in whole or part, imaged in fetal ultrasound imaging data generated during ultrasound scanning, the AI model processes the ultrasound imaging data on a line/sample basis, and the probability that a fetal anatomical feature is imaged in new ultrasound imaging is generated on a line/sample basis.

In some embodiments, the AI model is trained with one or more of the following: i) supervised learning; ii) previously labelled ultrasound image datasets; and iii) cloud stored data.

In some embodiments, at least one of following steps are additionally employed: i) automatically annotating one or more of the selected fetal anatomical features; ii) automatically captioning one or more of the selected fetal anatomical features; and iii) automatically labelling one or more of the selected fetal anatomical features, prior to generation of the multi-media product.

In some embodiments, the multi-media product comprises 3D representation of the selected fetal anatomical feature formed of a plurality of 2D ultrasound images.

In some embodiments, the fetal anatomical feature is selected from group consisting of in whole or part, of a fetus, heart, arm, hand, back, fingers, leg, face, head, torso, feet, toes and views and characteristics of the foregoing.

In a second broad aspect of the present disclosure, there is provided an ultrasound system for generating a multi-media product from fetal ultrasound images, comprising an ultrasound scanner configured to acquire a plurality of new ultrasound frames; a processor that is communicatively connected to the ultrasound scanner and configured to: process each new ultrasound frame of a plurality of new ultrasound frames against an artificial intelligence (“AI”) model, wherein said AI model is trained so that when the AI model is deployed, the computing device i) identifies and selects one or more fetal anatomical features, in whole or part, imaged in fetal ultrasound imaging data generated during ultrasound scanning, as part of a clinical exam of the fetus, wherein the selected one or more fetal anatomical features are visually appealing for entertainment and keepsake purposes and are not part of a clinical assessment of the health or growth of the fetus; ii) acquires the new ultrasound image during ultrasound scanning; iii) processes, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and iv) incorporates ultrasound images comprising the selected fetal anatomical features into the multi-media product.

In some embodiments of this system, a system additionally comprises a display device configured to display a multi-media product.

In some embodiments of this system, the AI model is trained with a plurality of training ultrasound images comprising labelled fetal biometric measurements, generated by one of a manual, semi automatic means or fully automatic means.

In some embodiments of this system, the AI model is trained with a plurality of training ultrasound images, comprising fetal anatomical features labeled and tagged from an identifier menu by one of a manual, semi automatic means or fully automatic means.

In some embodiments of this system, the fetal biometric measurements are selected from the group consisting of head circumference (HC), crown rump length (CRL), humerus length (HL), radius length (RL), femur length (FL), ulna length (UL), tibia length (TL), biparietal diameter (BPD), and abdominal circumference (AC), and wherein the method of training the AI model comprises identifying and labelling one or more fetal anatomical features in a fetal ultrasound image using the fetal biometric measurements.

In some embodiments of this system, the processor is additionally configured to perform at least one of following steps: i) automatically annotate one or more of the selected fetal anatomical features; ii) automatically caption one or more of the selected fetal anatomical features; and iii) automatically label one or more of the selected fetal anatomical features, prior to generation of the multi-media product.

In some embodiments of this system, the fetal anatomical feature is selected from group consisting of in whole or part, of a fetus, heart, arm, hand, back, fingers, leg, face, head, torso, feet, toes and views and characteristics of the foregoing.

In a third broad aspect of the present disclosure, there is provided a computer-readable media storing computer-readable instructions, for execution by at least one processor, wherein when the instructions are executed by the at least one processor, the at least one processor is configured to process a new ultrasound frame of a plurality of new ultrasound frames against an artificial intelligence (“AI”) model, wherein said AI model is trained so that when the AI model is deployed, the computing device i) identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus; ii) acquires the new ultrasound image during ultrasound scanning; iii) processes, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and iv) incorporates ultrasound images comprising the selected fetal anatomical features into the multi-media product.

What is claimed is:

1. A method of generating a multi-media product from fetal ultrasound images, during scanning of a fetus using an ultrasound scanner, comprising:
deploying an artificial intelligence (AI) model to execute on a computing device communicably connected to the ultrasound scanner, wherein the AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus;
acquiring, at the computing device, a new fetal ultrasound image during ultrasound scanning;
processing, using the AI model, the new fetal ultrasound image to identify and select the one or more fetal anatomical features, in whole or part, which are visually appealing for entertainment and keepsake purposes and are not part of a clinical assessment of the health or growth of the fetus (the “selected fetal anatomical features”); and
incorporating ultrasound images comprising the selected fetal anatomical features into the multi-media product.

2. The method of claim 1 wherein the AI model is trained with a plurality of training ultrasound images comprising labelled fetal biometric measurements, generated by one of a manual, semi automatic means or fully automatic means.

3. The method of claim 1 wherein the AI model is trained with a plurality of training ultrasound images, comprising fetal anatomical features labeled and tagged from an identifier menu by one of a manual, semi automatic means or fully automatic means.

4. The method of claim 1 wherein the new fetal ultrasound image is one or a combination of a live acquired fetal ultrasound image and a stored, previously acquired fetal ultrasound image.

5. The method of claim 2 wherein the fetal biometric measurements are selected from the group consisting of head circumference (HC), crown rump length (CRL), humerus length (HL), radius length (RL), femur length (FL), ulna length (UL), tibia length (TL), biparietal diameter (BPD), and abdominal circumference (AC), and wherein the method of training the AI model comprises identifying and labelling one or more fetal anatomical features in a fetal ultrasound image using the fetal biometric measurements.

6. The method of claim 1, wherein when identifying and selecting one or more fetal anatomical features, in whole or part, imaged in fetal ultrasound imaging data generated during ultrasound scanning, the AI model processes the fetal ultrasound imaging data on a per pixel basis, and the probability that a fetal anatomical feature is imaged in new ultrasound imaging is generated on a per pixel basis.

7. The method of claim 1 wherein when identifying and selecting one or more fetal anatomical features, in whole or part, imaged in fetal ultrasound imaging data generated during ultrasound scanning, the AI model processes the ultrasound imaging data on a per pixel basis, and the probability that a fetal anatomical feature is imaged in new ultrasound imaging is generated on a per pixel basis, and wherein, when deployed, an output of the AI model for a first pixel of the new ultrasound imaging data is used to corroborate the output of the AI model for a second pixel of the new ultrasound imaging data adjacent to the first pixel.

8. The method of claim 1, wherein when identifying and selecting one or more fetal anatomical features, in whole or part, imaged in fetal ultrasound imaging data generated during ultrasound scanning, the AI model processes the ultrasound imaging data on a line/sample basis, and the probability that a fetal anatomical feature is imaged in new ultrasound imaging is generated on a line/sample basis.

9. The method of claim 1 comprising training the AI model with one or more of the following: i) supervised learning; ii) previously labelled ultrasound image datasets; and iii) cloud stored data.

10. The method of claim 1 additionally comprising at least one of following steps: i) automatically annotating one or more of the selected fetal anatomical features; ii) automatically captioning one or more of the selected fetal anatomical features; and iii) automatically labelling one or more of the selected fetal anatomical features, prior to generation of the multi-media product.

11. The method of claim 1 wherein the multi-media product comprises 3D representation of the selected fetal anatomical feature formed of a plurality of 2D ultrasound images.

12. The method of claim 1 wherein the anatomical feature is selected from group consisting of in whole or part, of a fetus, heart, arm, hand, back, fingers, leg, face, head, torso, feet, toes and views and characteristics of the foregoing.

13. An ultrasound system for generating a multi-media product from fetal ultrasound images, comprising:
i) an ultrasound scanner configured to acquire a plurality of new ultrasound frames;
ii) a processor that is communicatively connected to the ultrasound scanner and configured to:
process each new ultrasound frame of a plurality of new ultrasound frames against an artificial intelligence (“AI”) model, wherein said AI model is trained so that when the AI model is deployed, the computing device identifies and selects one or more fetal anatomical features, in whole or part, imaged in fetal ultrasound imaging data generated during ultrasound scanning, as part of a clinical exam of the fetus, wherein the selected one or more fetal anatomical features are visually appealing for entertainment and keepsake purposes and are not part of a clinical assessment of the health or growth of the fetus;
acquire the new ultrasound image during ultrasound scanning;
process, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and
incorporating ultrasound images comprising the selected fetal anatomical features into the multi-media product.

14. The ultrasound system of claim 13 additionally comprising a display device configured to display a multi-media product.

15. The ultrasound system of claim 13 wherein the AI model is trained with a plurality of training ultrasound images comprising labelled fetal biometric measurements, generated by one of a manual, semi automatic means or fully automatic means.

16. The ultrasound system of claim 13 wherein the AI model is trained with a plurality of training ultrasound images, comprising fetal anatomical features labeled and tagged from an identifier menu by one of a manual, semi automatic means or fully automatic means

17. The ultrasound system of claim 13 wherein the fetal biometric measurements are selected from the group consisting of head circumference (HC), crown rump length (CRL), humerus length (HL), radius length (RL), femur length (FL), ulna length (UL), tibia length (TL), biparietal diameter (BPD), and abdominal circumference (AC), and wherein the method of training the AI model comprises identifying and labelling one or more fetal anatomical features in a fetal ultrasound image using the fetal biometric measurements.

18. The ultrasound system of claim 13 wherein the processor is additionally configured to perform at least one of following steps: i) automatically annotate one or more of the selected fetal anatomical features; ii) automatically caption one or more of the selected fetal anatomical features; and iii) automatically label one or more of the selected fetal anatomical features, prior to generation of the multi-media product.

19. The ultrasound system of claim 13 wherein the anatomical feature is selected from group consisting of in whole or part, of a fetus, heart, arm, hand, back, fingers, leg, face, head, torso, feet, toes and views and characteristics of the foregoing.

20. A computer-readable media storing computer-readable instructions, for execution by at least one processor, wherein when the instructions are executed by the at least one processor, the at least one processor is configured to:
process a new ultrasound frame of a plurality of new ultrasound frames against an artificial intelligence (“AI”) model, wherein said AI model is trained so that when the AI model is deployed, the computing device identifies and selects one or more fetal anatomical features, in whole or part, imaged in fetal ultrasound imaging data generated during ultrasound scanning as part of a clinical exam of the fetus, wherein the selected one or more fetal anatomical features are visually appealing for entertainment and keepsake purposes and are not part of a clinical assessment of the health or growth of the fetus;
acquire the new ultrasound image during ultrasound scanning;
process, using the AI model, the new fetal ultrasound image to identify and select one or more fetal anatomical features, in whole or part, which are visually appealing for entertainment and keepsake purposes and are not part of the clinical assessment of the health or growth of the fetus (the “selected fetal anatomical features”); and
incorporating ultrasound images comprising the selected fetal anatomical features into the multi-media product.
